(19) United States
(12) Patent Application Publication
(10) Pub. No.: US 2024/0218572 A1
(43) Pub. Date: Jul. 4, 2024

(54) APPARATUS AND METHOD FOR MANUFACTURING BIODEGRADABLE THREAD, AND BIODEGRADABLE THREAD MANUFACTURED THEREBY

(71) Applicant: HYUNDAEMEDITECH CO., LTD., Wonju-si (KR)

(72) Inventors: Mi Hee SONG, Wonju-si (KR); Chul PARK, Yongin-si (KR)

(21) Appl. No.: 17/907,250

(22) PCT Filed: Sep. 5, 2022
(86) PCT No.: PCT/KR2022/013236

§ 371 Date: Sep. 23, 2022

(30) Foreign Application Priority Data
Mar. 4, 2022 (KR) 10-2022-0027918

(51) Int. Cl. D02G 3/44; A61B 17/06; D02J 3/10

(52) U.S. Cl. CPC D02G 3/448; A61B 17/06; D02J 3/10; D10B 2331/00; D10B 2509/00

(57) ABSTRACT

Disclosed herein is an apparatus for manufacturing a biodegradable thread including: a thread supply unit for supplying a biodegradable thread; a cog forming unit for forming cogs on the outer surface of the biodegradable thread supplied by the thread supply unit; and a cog axially deforming unit for axially deforming the cogs of the biodegradable thread, which has the cogs formed by the cog forming unit, with respect to a virtual central line of the biodegradable thread.

### TECHNICAL FIELD

The present invention relates to an apparatus and a method for manufacturing a biodegradable thread, and a biodegradable thread manufactured thereby. More specifically, the present invention relates to a biodegradable thread manufacturing apparatus capable of smoothly forming cogs on a biodegradable thread, wherein the cogs are biased to one side with respect to a virtual central line of the biodegradable thread so as to partially protrude without returning to their original positions even if the biodegradable thread is wound, a biodegradable thread manufacturing method, and a biodegradable thread manufactured thereby.

### BACKGROUND ART

Recently, the facial plastic procedure is turning into a trend from a facial lifting era using a scalpel toward a lifting era using a melting thread without using the scalpel. The lifting procedure using the melting thread is a plastic procedure, for instance, for removing wrinkles formed on the face, the lower jaw, the neck, and the so on, and adopts the method of pulling wrinkles by inserting the biodegradable thread (suture) into the skin.

Such a lifting procedure method is performed through the steps of inserting a biodegradable thread into a needle-shaped cannula 1 (see FIG. 1), inserting the cannula, for instance, into the cheek of the face, and withdrawing the cannula while pressing the inserted biodegradable thread.

Such a procedure has been used widely since being excellent at wrinkle removal, being simple, and having few side effects in comparison with drug treatment like a Botox injection method.

The biodegradable thread is made of polyglycolic acid, polydioxanone, polyglycolate, and the like, which can be dissolved and absorbed into the skin. More specifically, polydioxanone is used for a biomedical purpose, especially, used as a suture. Such a biodegradable thread is usually decomposed and disappeared within six months, and thus, there is almost no foreign body reaction.

Meanwhile, as illustrated in FIG. 1, a biodegradable thread 10 includes spiniform cogs 11 which is diagonally formed on the surface of the biodegradable thread 10 to perform skin lifting more effectively. The cogs 11 are formed on the biodegradable thread 10 by a biodegradable thread manufacturing apparatus. The cogs 11 are regularly formed in the length direction and in the circumferential direction of the biodegradable thread 10.

For example, the cogs 11 are formed on the outer surface of the biodegradable thread 10 in the spiral direction at an angle of 45 degrees. The cogs 11 formed on the biodegradable thread 10 can take the tissue of the skin better, so that the biodegradable thread 10 can perform skin lifting of a desired area of the skin better.

However, the biodegradable thread 10 on which the cogs 11 are formed is stored in a rolled state. While being rolled, the cogs 11 are inserted into a recessed area 11a of the biodegradable thread 10. So, the lifting effect may be deteriorated since the cogs 11 are not spread out well. In order to prevent deterioration of the lifting effect, the cogs 11 must be spread out before a lifting procedure, and it may make an operator or a user inconvenient.

Therefore, people require development of an apparatus and a method for manufacturing a new biodegradable thread capable of smoothly forming cogs 11 on a biodegradable thread 10 and preventing cogs 11 from being reinserted into the recessed area 11a when the biodegradable thread 10 is wound on a roll, and a biodegradable thread manufactured by the apparatus and the method.

### DISCLOSURE

### Technical Problem

Accordingly, the present invention has been made in view of the above-mentioned problems occurring in the related art, and it is an object of the present invention to provide an apparatus and a method for manufacturing a biodegradable thread, and a biodegradable thread manufactured thereby, which can smoothly form cogs on the biodegradable thread, and make the cogs partially protrude without returning to their original positions even if the biodegradable thread is wound since the cogs are biased to one side with respect to a virtual central line of the biodegradable thread, thereby enhancing efficiency in a lifting procedure without performing additional work to spread the cogs when the biodegradable thread is applied to the skin or the like.

The aspects of the present disclosure are not limited to those mentioned above, and other aspects not mentioned herein will be clearly understood by those skilled in the art from the following description.

### Technical Solution

To accomplish the above-mentioned objects, according to the present invention, there is provided an apparatus for manufacturing a biodegradable thread including: a thread supply unit for supplying a biodegradable thread; a cog forming unit for forming cogs on the outer surface of the biodegradable thread supplied by the thread supply unit; and a cog axially deforming unit for axially deforming the cogs of the biodegradable thread, which has the cogs formed by the cog forming unit, with respect to a virtual central line of the biodegradable thread.

In an embodiment of the present invention, the apparatus for manufacturing a biodegradable thread further includes a thread rotating unit disposed between the thread supply unit and the cog forming unit and axially rotating the biodegradable thread supplied by the thread supply unit at a predetermined angle so as to form the cogs on the biodegradable thread in a spiral direction in the cog forming unit.

In an embodiment of the present invention, the apparatus for manufacturing a biodegradable thread further includes a winding unit disposed at the rear of the cog axially deforming unit to wind the biodegradable thread having the cogs axially deformed by the cog axially deforming unit.

In an embodiment of the present invention, the cog axially deforming unit includes: a deformation body; a heating member disposed inside the deformation body to heat the biodegradable thread inserted into the deformation body; a hitting member for hitting the cogs of the biodegradable thread softened by the heating member so that the cogs are biased to one side with respect to the virtual central line of the biodegradable thread; and a cooling member for cooling the biodegradable thread having the cogs hit by the hitting member.

In another aspect of the present invention, there is provided a method for manufacturing a biodegradable thread including: a thread supplying step of supplying a biodegradable thread by a thread supply unit; a cog forming step of forming cogs on the biodegradable thread supplied by the thread supply unit by using a cog forming unit; and an axially deforming step of axially deforming the cogs formed on the biodegradable thread by using a cog axially deforming unit with respect to the biodegradable thread.

Meanwhile, the biodegradable thread according to an embodiment of the present invention includes a plurality of cogs formed on the outer surface, wherein the plurality of cogs are formed to be biased to one side with respect to a virtual central line.

In an embodiment of the present invention, the biodegradable thread is heated till reaching a softening point section so as to be softened, external force is applied to the cogs so that the cogs are axially deformed to one side with respect to the virtual central line, and the biodegradable thread is cooled, so that the plurality of cogs are biased to one side with respect to the virtual central line.

Meanwhile, the plurality of cogs are formed through a molding process of applying pressure to the biodegradable thread. The molding process is performed in a vacuum state, and after the molding process, a cutting process using press punching is performed.

### Advantageous Effects

As described above, the apparatus and method for manufacturing a biodegradable thread, and the biodegradable thread manufactured thereby according to preferred embodiments of the present invention can smoothly form cogs on the biodegradable thread, and make the cogs partially protrude without returning to their original positions even if the biodegradable thread is wound since the cogs are biased to one side with respect to a virtual central line of the biodegradable thread, thereby enhancing efficiency in a lifting procedure without performing additional work to spread the cogs when the biodegradable thread is applied to the skin or the like.

### DESCRIPTION OF DRAWINGS

FIG. 1 is a view illustrating a state in which a biodegradable thread having cogs is mounted in a cannula.

FIG. 2 is a schematic diagram illustrating a biodegradable thread manufacturing apparatus according to an embodiment of the present invention.

FIG. 3 is a schematic diagram illustrating a cog axial deformation unit illustrated in FIG. 2.

FIG. 4 is a cross-sectional view taken along the line IV-IV of FIG. 3.

FIG. 5 is a schematic diagram of a biodegradable thread axially deformed by the cog axial deformation unit of FIG. 3.

FIG. 6 is a flow chart of a biodegradable thread manufacturing method of the biodegradable thread manufacturing apparatus of FIG. 2.

FIGS. 7 to 9 are views for depicting a biodegradable thread manufactured according to another embodiment of the present invention.

FIGS. 10 and 11 are views for depicting a biodegradable thread manufactured according to a further embodiment of the present invention.

### MODE FOR INVENTION

Advantages and features of the present disclosure and methods accomplishing the advantages and features will become apparent from the following detailed description of exemplary embodiments with reference to the accompanying drawings. However, the present invention is not limited to exemplary embodiment disclosed herein but will be implemented in various forms. The exemplary embodiments are provided so that the present invention is completely disclosed, and a person of ordinary skilled in the art can fully understand the scope of the present invention. Therefore, the present invention will be defined only by the scope of the appended claims. In addition, like reference numerals designate like elements throughout the specification.

Hereinafter, preferred embodiments according to the present invention will be described in detail with reference to the drawings.

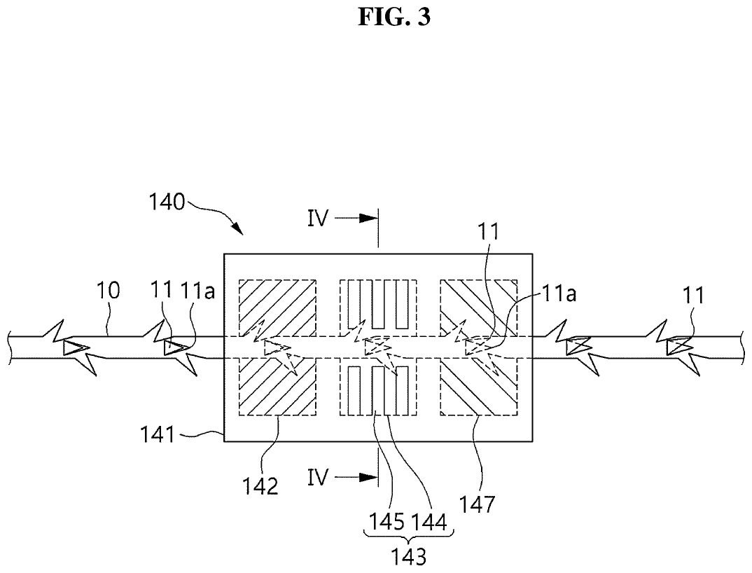

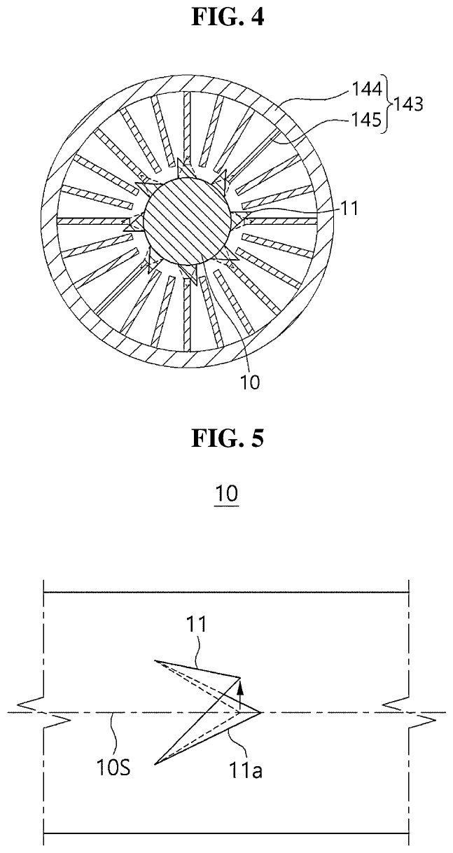

FIG. 2 is a schematic diagram illustrating a biodegradable thread manufacturing apparatus according to an embodiment of the present invention, FIG. 3 is a schematic diagram illustrating a cog axial deformation unit illustrated in FIG. 2, FIG. 4 is a cross-sectional view taken along the line IV-IV of FIG. 3, and FIG. 5 is a schematic diagram of a biodegradable thread axially deformed by the cog axial deformation unit of FIG. 3.

As illustrated in the drawings, a biodegradable thread manufacturing apparatus 100 according to an embodiment of the present invention is an apparatus for manufacturing a biodegradable thread 10 (a suture) on which cogs 11 are formed, and includes: a thread supply unit 110 for supplying the biodegradable thread 10; a thread rotating unit 120 for axially rotating the biodegradable thread 10 supplied by the thread supply unit 110 to a predetermined angle; a cog forming unit 130 for forming cogs 11 on the outer surface of the biodegradable thread 10; a cog axially deforming unit 140 for axially deforming the cogs 11 formed on the biodegradable thread 10 to be biased to one side with respect to a virtual central line 10S (See FIG. 5) of the biodegradable thread; and a winding unit 150 for winding the biodegradable thread 10 having the axially deformed cogs 11.

Here, the virtual central line 10S may be a virtual line extending in the longitudinal direction of the biodegradable thread 10, but is not limited thereto. That is, the cogs 11 may have one of various forms twisted to one side at an angle without a bilaterally symmetric form.

Through such a configuration, the present invention can smoothly form cogs 11 on the biodegradable thread 10, and make the cogs 11 partially protrude without returning to their original positions even if the biodegradable thread 10 is wound since the cogs are biased to one side with respect to a virtual central line 10S of the biodegradable thread 10, thereby enhancing efficiency in a lifting procedure without performing additional work to spread the cogs 11 when the biodegradable thread 10 is applied to the skin or the like.

Although not illustrated in detail, the thread supply unit 110 according to the present embodiment is provided in a roll type in which the biodegradable thread 10 is wound, and provides the biodegradable thread 10, which is a raw material, at a predetermined speed by rotation.

In other words, the biodegradable thread 10 of the present embodiment may be made of polydioxanone (PDO) which is biodegradable when time elapses even if the biodegradable thread 10 is inserted into the skin or the like. As will be described later, a plurality of spiniform cogs 11 are provided to increase the binding force with the skin tissue.

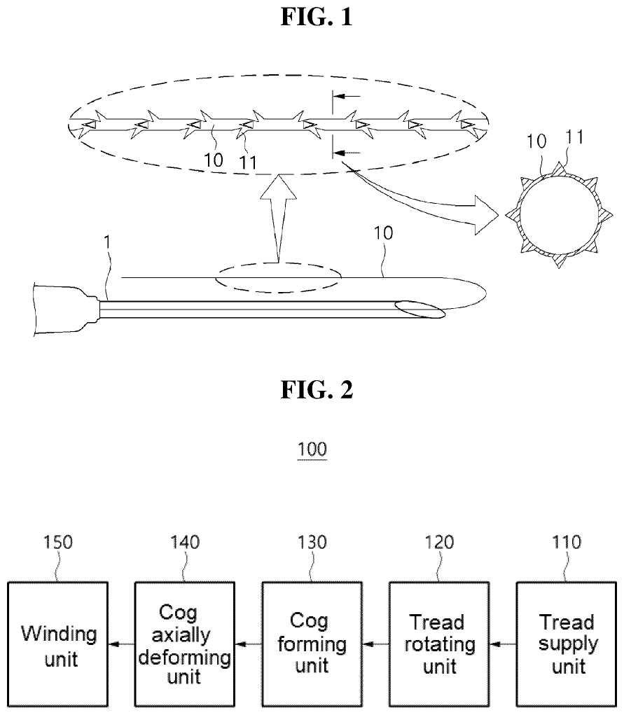

In other words, when the biodegradable thread 10 is inserted into the skin tissue, such as the cheek of the face, using a cannula 1 (See FIG. 1), the biodegradable thread 10 is increased in closeness with the skin tissue due to the cogs 11, thereby providing excellent lifting effect.

However, the material of the biodegradable thread 10 is not limited thereto, and for example, the biodegradable thread 10 may be made of polyglycolic acid, a polyglycolate, or the like, and the shape of the cogs 11 may also be varied.

Meanwhile, although not illustrated in detail, the thread rotating unit 120 rotates the thread provided from the thread supply unit 110 at a predetermined angle to form cogs 11 as set on the biodegradable thread 10 by the cog forming unit 130.

For example, the thread rotating unit 120 rotates the biodegradable thread 10 by 45 degrees at a set time interval, and thus, as illustrated in FIGS. 1 and 3, cogs 11 are formed on the outer surface of the biodegradable thread 10 at an angle of 45 degrees in a spiral direction.

However, the degree of rotation of the thread rotating unit 120 is not limited thereto, and the thread rotating unit 120 may have the degree of rotation of, for example, 90 degrees or 180 degrees.

Meanwhile, although not illustrated in detail, the cog forming unit 130, which forms cogs 11 on the biodegradable thread 10, may be provided as a cutter or a cutting blade for partially cutting the outer surface of the biodegradable thread 10.

As described above, the cog forming unit 130 is interlocked with the thread rotating unit 120 to form cogs 11 on the biodegradable thread 10. As illustrated in FIG. 1, the cogs 11 are provided on the outer surface of the biodegradable thread 10 at 45 degrees in the spiral direction, thereby increasing the degree of closeness with the skin during a procedure to promote collagen formation.

However, conventionally, cogs 11 are formed on a biodegradable thread 10 and rolled into a roll type, and then, the biodegradable thread 10 is unrolled when being used for a procedure. In this case, the cogs 11 may be inserted into an original cut-out region 11a of the biodegradable thread 10 while the biodegradable thread 10 is wound. So, there is inconvenience to withdraw the cogs 11 from a body of the biodegradable thread 10 for a procedure.

Accordingly, in the present embodiment, the cogs 11 formed on the biodegradable thread 10 are biased to one side, thereby preventing the cogs 11 from being inserted into the original cut-out area 11a even if the biodegradable thread 10 is wound. So, the manufacturing apparatus 100 according to the present embodiment further includes a cog axially deforming unit 140 to deform the position of the cogs 11.

As illustrated in FIG. 3, the cog axially deforming unit 140 includes: a cylindrical deformation body 141; a heating member 142 provided in the cylindrical deformation body 141 to heat and soften the biodegradable thread 10; a hitting member 143 for hitting the cogs 11 of the biodegradable thread 10 softened by the heating member 142 to make the cogs 11 biased to one side with respect to the virtual central line 10S of the biodegradable thread 10, namely, to deform the cogs 11; and a cooling member 147 for cooling and hardening the biodegradable thread 10 having the cogs 11 biased to one side.

First, as illustrated in FIG. 3, the heating member 142 is provided in a hollow cylindrical shape surrounding the outer surface of the biodegradable thread 10, and heats the biodegradable thread 10 to soften the biodegradable thread 10, and in this instance, the softening point of the biodegradable thread 10 is in the range of 70 to 90° C.

As described above, because the biodegradable thread 10 and the cogs 11 are softened when the heating member 142 heats the biodegradable thread 10. Accordingly, when the hitting member 143 pushes or hits the cogs 11 to one side, the cogs 11 are biased to one side as illustrated in FIGS. 4 and 5. That is, the cogs 11 are axially deformed with respect to the virtual central line 10S of the biodegradable thread 10.

Referring to FIGS. 3 and 4, the hitting member 143 includes a perforated portion 144 through which the biodegradable thread 10 passes, and a plurality of prong portions 145 arranged along the inner wall of the perforated portion 144. The prong portion 145 is made of a flexible material and has a length such that the end thereof gets in contact with the cog 11.

Here, the perforated portion 144 is rotated in place. When the perforated portion 144 rotates, the prong portions 145 in the perforated portion 144 rotate together to hit the cogs 11 to one side. More specifically, the prong portions 145 can push the cogs 11 to one side as if sweeping. Therefore, as indicated by the arrow in FIG. 5, the cogs 11 are axially deformed at the original positions.

In other words, the cogs 11 are biased to one side with respect to the virtual central line 10S of the biodegradable thread 10 by the hitting of the hitting member 143. So, since the cogs are not reinserted into the cut-out region 11a even if the biodegradable thread 10 is wound. Therefore, it is possible to prevent inconvenience of spreading the cogs 11 again during the procedure.

As described above, the hitting member 143 hits the softened cogs 11 of the biodegradable thread 10 to axially deform the cogs 11, and then, the cooling member 147 cools the biodegradable thread 10 below the softening point so as to harden the biodegradable thread 10. That is, the deformation position of the cogs 11 with respect to the biodegradable thread 10 can be made form.

Meanwhile, although not illustrated, the prong portions 145 of the hitting member 143 are formed long such that the ends of the prong portions get in contact with the outer surface of the biodegradable thread 10. In this case, the prong portions 145 are rotated by the rotation of the perforated portion 144 so that the cogs 11 are axially deformed and the ends of the prong portions 145 form scratches on the outer surface of the biodegradable thread 10.

Therefore, in a case in which the biodegradable thread 10 is applied to the skin, a contact force between the biodegradable thread 10 and the skin is increased due to not only the cogs 11 but also the scratched portions, thereby maximizing the effect of a lifting procedure.

The winding unit 150 is disposed at the rear of the cog axially deforming unit 140 to wind the biodegradable thread 10 having the cogs 11 axially deformed by the cog axially deforming unit 140. The winding unit 150 may be detachably coupled to the manufacturing apparatus 100 according to the present embodiment, and may be supplied in a state on which the biodegradable thread 10 is wound.

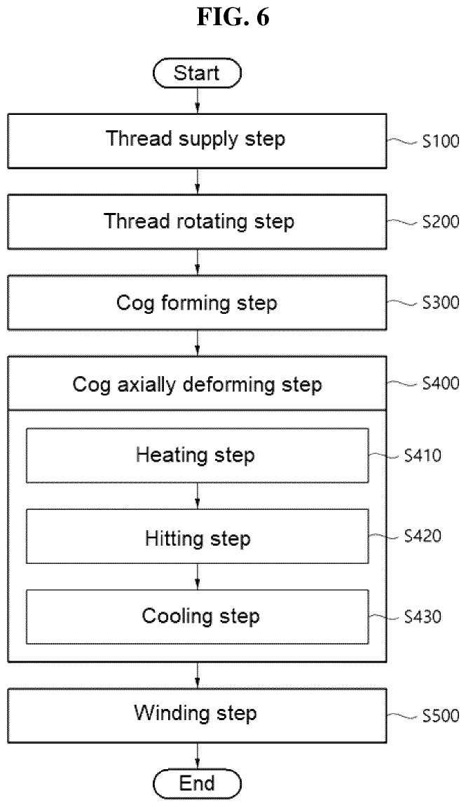

Meanwhile, a biodegradable thread manufacturing method of the biodegradable thread manufacturing apparatus according to the present invention will be described with reference to FIG. 6.

FIG. 6 is a flow chart of the biodegradable thread manufacturing method of the biodegradable thread manufacturing apparatus of FIG. 2.

As illustrated therein, the biodegradable thread manufacturing method according to the present embodiment includes: a thread supply step S100 of supplying a biodegradable thread 10 by a thread supply unit 110; a thread rotating step S200 of rotating the biodegradable thread 10 at a predetermined angle by using the thread rotating unit 120; a cog forming step S300 of forming cogs 11 on the biodegradable thread 10 by the cog forming unit 130; an axially deforming step S400 of axially deforming the cogs 11 formed on the biodegradable thread 10 with respect to the biodegradable thread 10 by using the cog axially deforming unit 140; and a winding step S500 of winding the biodegradable thread 10 by using the winding unit 150.

As described above, in the thread supply step S100, the biodegradable thread 10, which is a raw material, is supplied. In the thread rotating step S200, the supplied biodegradable thread 10 is rotated at the predetermined angle, for instance, at 45 degrees, in one direction (in the clockwise direction or in the counterclockwise direction). After that, in the cog forming step S300, cogs 11 are formed on the rotating biodegradable thread 10. Therefore, as illustrated in FIG. 3, the cogs 11 are formed on the biodegradable thread 10 in the spiral direction at intervals of 45 degrees.

The cog axially deforming step S400, as illustrated in FIG. 6, includes: a heating step S410 of heating the biodegradable thread 10 by using the heating member 142 to heat the biodegradable thread 10 up to a softening point; a hitting step S420 of hitting the cogs 11 of the softened biodegradable thread 10 by the hitting member 143 to be biased to one side, namely, to axially deform the cogs; and a cooling step S430 of cooling the biodegradable thread 10 having the axially deformed cogs 11 by using the cooling member 147.

As illustrated in FIGS. 4 and 5, the shaft-deformed cogs 11 can be formed with respect to the imaginary center line (10S) of the biodegradable thread 10, as illustrated in FIGS. 4 and 5, and thus the biodegradable thread 10 can be smoothly wound by the winding unit 150 in the winding step (S500), and the protrusion of the cogs 11 with respect to the biodegradable thread 10 can be maintained even when the biodegradable thread 10 is released during a subsequent procedure, thereby smoothly and efficiently performing the procedure.

As described above, through the axially deforming step S400, the cogs 11 axially deformed with respect to the virtual central line 10S of the biodegradable thread 10 are formed. Therefore, the biodegradable thread 10 can be smoothly wound by the winding unit 150 in the winding step S500, and the cogs 11 can maintain the protruding state with respect to the biodegradable thread 10 even if the biodegradable thread 10 is unwound during a procedure, so that the procedure can be performed smoothly and effectively.

As described above, the present invention can smoothly form cogs 11 on the biodegradable thread 10, and make the cogs 11 partially protrude without returning to their original positions even if the biodegradable thread 10 is wound since the cogs 11 are biased to one side with respect to a virtual central line 10S of the biodegradable thread 10, thereby enhancing efficiency in a lifting procedure without performing additional work to spread the cogs 11 when the biodegradable thread 10 is applied to the skin or the like.

With reference to FIGS. 1 to 8, the method for manufacturing a biodegradable thread having cogs which are formed to be biased to one side with respect to the virtual central line through the cog axially deforming step S400 including the heating step, the hitting step, and the cooling step is described, but the present invention is not limited thereto.

For example, according to another embodiment of the present invention, a plurality of cogs formed to be biased to one side may be formed through a molding process and a cutting process, so that the cogs may have various shapes.

Here, the molding process means to change a shape of the corresponding part by applying pressure to a part of the biodegradable thread, and the cutting process means to process the final shape of the cogs by using press punching.

Meanwhile, the molding process may be performed in a vacuum state. In a case in which molding is performed in a vacuum state, pressure is applied to the biodegradable thread maintaining temperature within about 40 degrees to deform the biodegradable thread, and the molding process is performed in a condition that oxygen and moisture are removed, thereby minimizing an internal damage of the biodegradable thread.

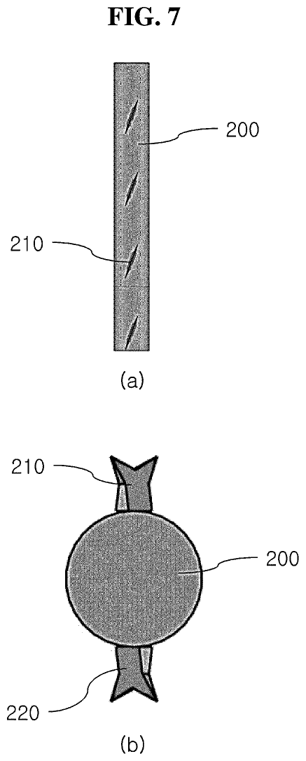

FIGS. 7 to 9 are diagrams for depicting a biodegradable thread manufactured according to another preferred embodiment of the present invention. Descriptions of the present embodiment having the same parts as the embodiment described referring to FIGS. 1 to 8 will be omitted.

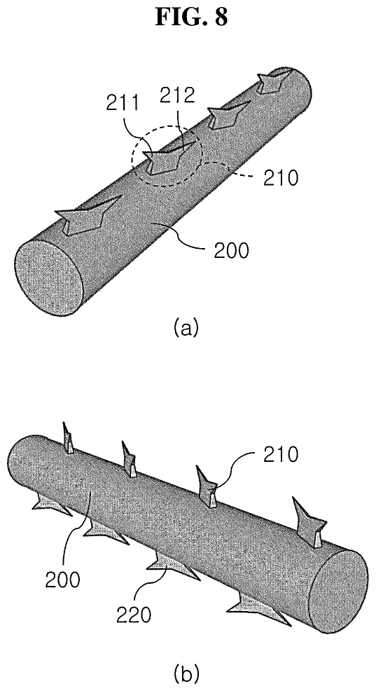

Referring to FIGS. 7 and 8, through the molding process and the cutting process as described above, a plurality of cogs 210 which are formed to be biased to one side with respect to a virtual central line extending in the longitudinal direction of a biodegradable thread 200 and to have a twisted angle are formed on the outer surface of the biodegradable thread 200.

Meanwhile, the cogs formed on the biodegradable thread 200 may have angles twisted in different directions, and for instance, upper cogs 210 and lower cogs 220 are biased to one side with respect to the virtual central line, but are twisted in different directions.

In addition, the cogs 210 formed in the biodegradable thread 200 may have a shape in which the end portions are split on both sides. For instance, the end portions of the cogs 210 may have two or more sharp protrusions 211 and 212.

According to the shape and structure of the cogs 210 and 220 as described with reference to FIGS. 7 and 8, an area of the skin pulled by the cogs 210 and 220 of the biodegradable thread 200 in the skin is increased, and especially, the cogs 210 and 220 can pull the skin widely not only in the longitudinal direction of the biodegradable thread 200 but also in the direction crossing the longitudinal direction of the biodegradable thread 200, thereby enhancing the skin lifting effect of the biodegradable thread 200.

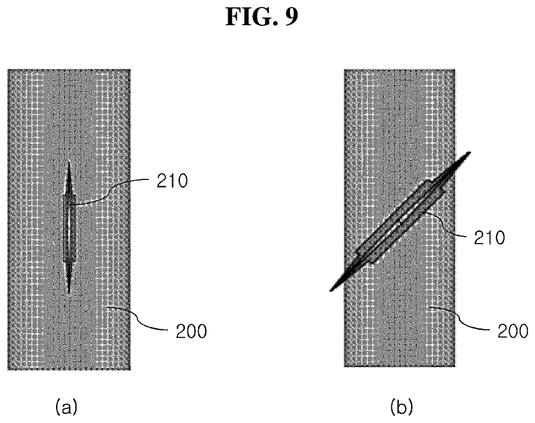

For example, as illustrated in FIG. 9(a), in a case in which the cogs 210 are formed in the longitudinal direction of the biodegradable thread 200, the area of the skin pulled is limited to the longitudinal direction of the biodegradable thread 200.

On the other hand, as illustrated in FIG. 9(b), in a case in which the cogs 210 are formed to be twisted at a predetermined angle with respect to the longitudinal direction of the biodegradable thread 200, the area of the skin pulled is further increased, thereby improving the lifting effect of pulling the skin.

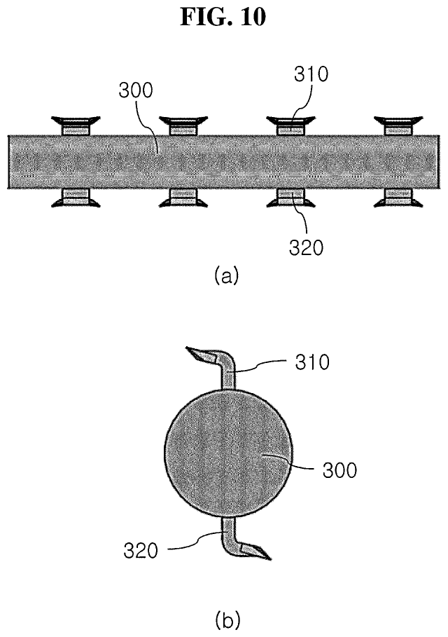

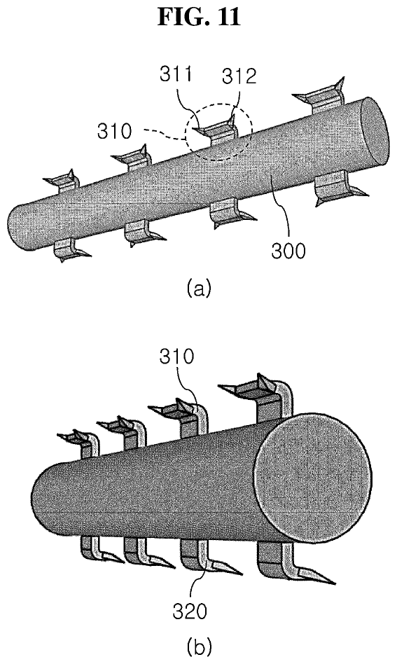

FIGS. 10 and 11 are views for depicting a biodegradable thread manufactured according to a further embodiment of the present invention. Descriptions of the present embodiment having the same parts as the embodiment described referring to FIGS. 1 to 9 will be omitted.

Referring to FIGS. 10 and 11, cogs 310 are formed on a biodegradable thread 300 through the molding process and the cutting process as described above. After that, pressure is applied to end portions of the cogs 310 in one direction so that the end portions of the cogs 310 are bent to be biased to one side with respect to a virtual central line. Accordingly, the middle portion of the cog 310 may have a rounded shape.

Meanwhile, the end portions of the cogs 310 formed on the biodegradable thread 300 are bent in different directions. For instance, upper cogs 310 and lower cogs 320 are bent to one side with respect to the virtual central line, but the end portions of the upper cogs 310 and the end portions of the lower cogs 320 are bent in different directions.

In addition, the cogs 210 formed in the biodegradable thread 200 may have a shape in which the end portions are split on both sides. For instance, the end portions of the cogs 210 may have two or more sharp protrusions 311 and 312.

FIGS. 10 and 11 show the structure of a biodegradable thread to further improve the skin-pulling effect of the cogs. The cog 310 is bent in a rounded shape, and the end of the cog 310 is split on both sides to form the protrusions 311 and 312.

As an embodiment of a biodegradable thread manufacturing method for forming the cogs having the above-described structure, both cogs are formed through a molding process and a press process, and then a molding process can be used again to bend the cog in one direction.

Compared to forming a molding cog by manufacturing a molding mold and a press mold and sequentially proceeding the molding process and the press process, the cog, which is curved to one side and has a rounded shape as described above, can improve the biodegradable thread's structure and its effects by adding a mold and a process.

More specifically, by a first molding process of pressing a circular biodegradable thread to spread it laterally, a pressing process of striking one or both ends of the biodegradable thread by hitting the unfolded part of the biodegradable thread with a press mold, and a second molding process of bending by pressing again the unfolded part with a molding mold, the cog of the biodegradable thread as described with reference to FIGS. 10 and 11 can be formed.

The molding process using the molding mold is for deforming the shape of a part of the biodegradable thread, and the pressing process using the press mold for cutting a part of the biodegradable thread, and protrusions 311 and 312 split on both sides at the end of the cog 310 may be formed by the pressing process.

According to structures of the biodegradable thread and the cogs as described with reference to FIGS. 10 and 11, as the cog is bent to one side and has a curved shape, when inserting the biodegradable thread into an injection needle, the biodegradable thread can be inserted smoothly even into a thinner injection needle. Thereby the pain that may occur when the needle is inserted into the skin can be reduced.

In addition, according to the structures of the biodegradable thread and the cogs as described with reference to FIGS. 10 and 11, the cog is bent in both diagonal directions of the biodegradable thread, so it can pull the skin into the skin and keep it in a pulled state in a larger area, thereby the performance of the lifting thread can be improved with more stability.

it is better to pull the skin in the skin and keep it in the pulled state. It is made with a large area, which can improve the function of the lifting seal with more stability.

Although a specific embodiment according to the present invention has been described above, various modifications are possible within the scope of the present invention without departing from the scope of the present invention. Therefore, the scope of the present invention should not be limited to the described embodiments, and the scope of the present invention should be determined not only by the scope of the claims which will be described below, but also the scope of the claims.

The foregoing embodiments and advantages are merely exemplary and are not to be construed as limiting the present invention. The present teaching can be readily applied to other types of apparatuses. The description of the foregoing embodiment is intended to be illustrative, and not to limit the scope of the claims. Many alternatives, modifications, and variations will be apparent to those skilled in the art.

1. An apparatus for manufacturing a biodegradable thread comprising:
a thread supply unit for supplying a biodegradable thread;
a cog forming unit for forming cogs on the outer surface of the biodegradable thread supplied by the thread supply unit; and
a cog axially deforming unit for axially deforming the cogs of the biodegradable thread, which has the cogs formed by the cog forming unit, with respect to a virtual central line of the biodegradable thread.

2. The apparatus according to claim 1, further comprising:
a thread rotating unit disposed between the thread supply unit and the cog forming unit and axially rotating the biodegradable thread supplied by the thread supply unit at a predetermined angle so as to form the cogs on the biodegradable thread in a spiral direction in the cog forming unit.

3. The apparatus according to claim 1, further comprising:
a winding unit disposed at the rear of the cog axially deforming unit to wind the biodegradable thread having the cogs axially deformed by the cog axially deforming unit.

4. The apparatus according to claim 1, wherein the cog axially deforming unit comprises:
a deformation body;
a heating member disposed inside the deformation body to heat the biodegradable thread inserted into the deformation body;
a hitting member for hitting the cogs of the biodegradable thread softened by the heating member so that the cogs are biased to one side with respect to the virtual central line of the biodegradable thread; and
a cooling member for cooling the biodegradable thread having the cogs hit by the hitting member.

5. The apparatus according to claim 4, wherein the hitting member comprises:
a perforated portion through which the biodegradable thread passes; and
a plurality of prong portions arranged along the inner wall of the perforated portion,
wherein the plurality of prong portions hits the cogs by the rotation of the perforated portion to axially deform the cogs to one side.

6. The apparatus according to claim 5, wherein the prong portions are formed such that end portions of the prong portions get in contact with the outer surface of the biodegradable thread, the end portions of the prong portions forms scratches on the outer surface of the biodegradable thread by the rotation of the perforated portion.

7. The apparatus according to claim 5, wherein the plurality of prong portions are made of a flexible material.

8. The apparatus according to claim 5, wherein the biodegradable thread is made of polydioxanone (PDO) having a softening point ranging from 70 to 90° C.,
wherein the heating member heats the polydioxanone in a section of the softening point to soften the polydioxanone, and
wherein the cooling member cools the polydioxanone below the softening point to harden the polydioxanone.

9. A method for manufacturing a biodegradable thread comprising:
a thread supplying step of supplying a biodegradable thread by a thread supply unit;
a cog forming step of forming cogs on the biodegradable thread supplied by the thread supply unit by using a cog forming unit; and
an axially deforming step of axially deforming the cogs formed on the biodegradable thread by using a cog axially deforming unit with respect to the biodegradable thread.

10. The method according to claim 9, further comprising:
a thread rotating step of rotating the biodegradable thread supplied from the thread supply unit at a predetermined angle by using a thread rotating unit so that the cogs are formed on the biodegradable thread in a spiral direction in the cog forming step, wherein the thread rotating step is performed between the thread supply step and the cog forming step.

11. The method according to claim 9, further comprising:
a winding step of winding the biodegradable thread, which has the cogs axially deformed in the axially deforming step, by a winding unit.

12. The method according to claim 9, wherein the axially deforming step comprises:
a heating step of heating the biodegradable thread by a heating member;
a hitting step of hitting the cogs of the biodegradable thread, which is softened by the heating step, by a hitting member so that the cogs are axially deformed to one side with respect to a virtual central line of the biodegradable thread; and
a cooling step of cooling the biodegradable thread, which has the cogs axially deformed by the hitting step, by a cooling member.

13. The method according to claim 12, wherein the biodegradable thread is made of polydioxanone (PDO) having a softening point ranging from 70 to 90° C.,
wherein in the heating step, the heating member heats the polydioxanone in a section of the softening point to soften the polydioxanone, and
wherein in the cooling step, the cooling member cools the polydioxanone below the softening point to harden the polydioxanone.

14. A biodegradable thread having a plurality of cogs formed on the outer surface, wherein the plurality of cogs are formed to be biased to one side with respect to a virtual central line.

15. The biodegradable thread according to claim 14, wherein the biodegradable thread is heated till reaching a softening point section so as to be softened, external force is applied to the cogs so that the cogs are axially deformed to one side with respect to the virtual central line, and the biodegradable thread is cooled, so that the cogs are biased to one side with respect to the virtual central line.

16. The biodegradable thread according to claim 14, wherein the plurality of cogs are formed through a molding process of applying pressure to the biodegradable thread.

17. The biodegradable thread according to claim 15, wherein the molding process is performed in a vacuum state, and
wherein after the molding process, a cutting process using press punching is performed.
